(19) United States
(12) Patent Application Publication
(10) Pub. No.: US 2011/0077405 A1
(43) Pub. Date: Mar. 31, 2011

(54) PROCESS FOR PREPARATION OF ENANTIOMERICALLY PURE (S)-1-PHENYI-1,2,3,4- TETRAHYDROISOQUINOLINE

(76) Inventors: Oliwia Zegrocka-Stendel, Lomianki (PL); Joanna Zagrodzka, Warszawa (PL); Marta Laszcz, WarsZawa (PL)

(21) Appl. No.: 12/993,874

(22) PCT Filed: May 22, 2009
(86) PCT No.: PCT/PL2009/000053

§ 371 Date: Dec. 1, 2010

(30) Foreign Application Priority Data
May 23, 2008 (PL) 385264

(51) Int. Cl. C07D 453/02; C07D 217/02

(52) U.S. Cl. 546/137; 546/144

(57) ABSTRACT

Process for preparation of (S)-1-phenyl-1,2,3,4-tetrahydroisoquinoline wherein 1-phenyl-1,2,3,4-tetrahydroisoquinoline is reacted with D-(−)-tartaric acid in a solvent system comprising of methanol and water, preferably at 3.3:1 to 1:1 volume ratio, the crystallization mixture is left for crystallization and (S)-1-phenyl-1,2,3,4-tetrahydroisoquinoline is released from obtained crystalline diastereoisomeric salt according to standard procedures. (S)-1-Phenyl-1,2,3,4-tetrahydroisoquinoline is the intermediate in enantiomeric synthesis of solifenacin.

### CROSS-REFERENCE TO RELATED APPLICATIONS

The present Application is a U.S. national phase of PCT/PL2009/000053 filed on May 22, 2009 (“PCT Application”), which claims priority from Polish Application No. 385264 filed on May 23, 2008, both of which are hereby incorporated by reference in their entirety into the present Application.

### FIELD OF THE INVENTION

The invention relates to the process for preparation of enantiomerically pure (S)-1-phenyl-1,2,3,4-tetrahydroisoquinoline, which is the intermediate in the synthesis of important pharmaceutical substances, including solifenacin.

Solifenacin, (R)-3-quinuclidinol (1S)-1-phenyl-1,2,3,4-tetrahydroisoquinolin-2-carboxylate (IUPAC name: 1-azabicyclo[2.2.2]oct-8-yl (1S)-1-phenyl-3,4-dihydroisoquinoline-2-carboxylate), is a competitive selective M3 muscarinic receptor antagonist. Solifenacin succinate is the active substance of Vesicare®, licensed for the treatment of overactive bladder symptoms of urge urinary incontinence, urgency and urinary frequency.

### BACKGROUND OF THE INVENTION

Preparation of solifenacin as a racemic mixture or active enantiomer (1S, 3R′) can be accomplished following one out of two possible synthetic methods. The first synthetic approach is based on the reaction of quinuclidinol and carbamoyl derivative of 1-phenyl-1,2,3,4-tetrahydroisoquinoline with good leaving group. The second one comprises the condensation of 1-phenyl-1,2,3,4-tetrahydroisoquinoline with activated quinucidinol derivative, for example chloroformate or carbonate derivative. In EP 0801067 B1 and WO 2005/105795 among good leaving groups chloride anion, lower alkoxides, phenoxide, 1H-imidazol-1-yl, 2,5-dioxopyrrolidin-1-yloxy and 3-methyl-1H-imidazol-3-ium-1-yl groups are mentioned.

In J. Med Chem., 2005, 48 (21), 6597-6606, solifenacin was prepared in transestrification reaction of (R)-quinuclidinol and ethyl (S)-1-phenyl-1,2,3,4-tetrahydroisoquinoline-2-carboxylate. This optically active intermediate was obtained in the prior step from (S)-1-phenyl-1,2,3,4-tetrahydroisoquinoline and ethyl chloroformate in the presence of potassium carbonate.

Regardless of the chosen methodology, (S)-1-phenyl-1,2,3,4-tetrahydroisoquinoline (Formula 1) is the crucial intermediate in enantioselective synthesis of solifenacin (Formula 2).

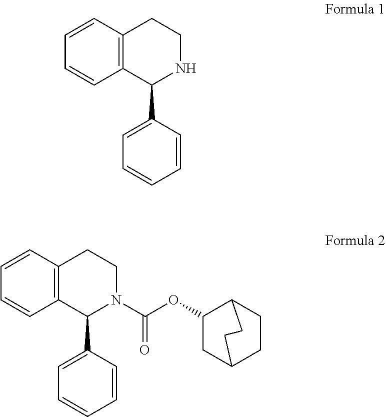

Preparation of enantiomerically pure (S)-1-phenyl-1,2,3,4-tetrahydroisoquinoline via enantiomeric resolution of racemic mixture with L-(+)-tartaric acid is known from the literature (Monach. Chem. 1929, 5354, 956-962).

In J. Med. Chem., 2005, 48 (21), 6597-6606 the resolution of 1-phenyl-1,2,3,4-tetrahydroisoquinoline racemic mixture onto pure enantiomers is described. This method comprises formation of diastereoisomeric salts with L-(+)-tartaric acid in ethanol, followed by recrystallization of the obtained (−)-tartarate from water. (S)-1-Phenyl-1,2,3,4-tetrahydroisoquinoline is liberated from diastereoisomeric salt upon treatment with sodium hydroxide aqueous solution, extraction with ethyl acetate, condensation of organic layer and recrystallization of collected crystals from hexane. The enantiomeric purity of obtained product was not given.

Experimental trials to employ hereinbefore described procedures in L-(+)-tartaric acid assisted enantiomeric resolution in ethanol, were unsuccessful. As a result either the isomeric mixture of different ratio or pure (R) enantiomer were obtained.

International patent application publication WO 2008/019055 discloses resolution of racemic 1-phenyl-1,2,3,4-tetrahydroisoquinoline with D-(−)-tartaric acid in isopropanol, optionally in a mixture with water, or in ethyl acetate. Examples of this publication comprise only (S)-1-phenyl-1,2,3,4-tetrahydroisoquinoline tartrate formation step; neither isolation procedure of (S) isomer nor total yield of this process were revealed. Optical purity of obtained (S)-1-phenyl-1,2,3,4-tetrahydroisoquinoline was not experimentally proved, though authors of this publication claimed it was at least 98%. The intermediate of declared level of enantiomeric purity is not suitable to be used in the synthesis of solifenacin, parameters of which must meet the requirements for authorized medicines. There is also a danger that racemisation on a chiral centre may occur, affecting decrease of optical purity of the final product, during the process of optically active base release from its salt under basic conditions. Following this procedure, to obtain optically active product of high enantiomeric purity, additional steps for (S)-1-phenyl-1,2,3,4-tetrahydroisoquinoline enrichment with (S) enantiomer would be required.

The limitations of described above enantiomeric resolution methods in industrial scale production process are low selectivity, usage of expensive optically active acids and tax excised solvents (eg. ethyl alcohol), as well as the partial loss of starting material resulted from racemisation and recycling to salt formation step.

Hence, there was a need to develop reproducible and selective process for preparation of (S)-1-phenyl-1,2,3,4-tetrahydroisoquinoline, which product would be characterized by high enantiomeric purity and high chemical yield close to theoretical one. These assumptions are necessary to fulfill to make the optical resolution method of racemic 1-phenyl-1,2,3,4-tetrahydroisoquinoline useful in a big laboratory or industrial scales.

### DISCLOSURE OF THE INVENTION

It was proved, these goals can be reached by resolving racemic mixture of 1-phenyl-1,2,3,4-tetrahydroisoquinoline due to diastereoisomeric salt formation with D-(−)-tartaric acid in a special selected solvent system according to the present invention.

The background of the invention relates to the discovery of phenomenon, that in the process of resolution of 1-phenyl-1,2,3,4-tetrahydroisoquinoline, salt enriched with (S)-enantiomer is formed, which shows very low solubility in alcohols and water, even at elevated temperatures. Optional additional crystallization necessary for enantiomeric purity increase, would be accompanied with the release of amine from its enantiomerically enriched salt and, as the result, the necessity of using of additional amount of D-(−)-tartaric acid for the salt formation.

Unexpectedly it was discovered by the present Inventors, that high crystallization selectivity of 1-phenyl-1,2,3,4-tetrahydroisoquinoline diastereoisomeric salt with D-(−)-tartaric acid may be accomplished in a solvent system which comprises methanol as the main solvent of choice in combination with a co-solvent. While using the said solvent/co-solvent system, crystallization selectivity of diastereoisomeric salt, consisting of desired (S) enantiomer, is increased and pure compound in high chemical yield is obtained.

The invention relates to the process for preparation of (S)-1-phenyl-1,2,3,4-tetrahydroisoquinoline due to resolution of optically active diastereoisomeric salts. The process is characterized in that 1-phenyl-1,2,3,4-tetrahydroisoquinoline is reacted with D-(−)-tartaric acid in a solvent system, consisting of methanol and water, the crystallization mixture is left for crystallization, and (S)-1-phenyl-1,2,3,4-tetrahydroisoquinoline is released from crystalline diastereoisomeric salt according to standard procedures.

In the preferred embodiment of the invention, the mixture of solvents used consists of at least 50% (v/v) of methanol, more preferably methanol and water at 3.3:1 to 2:1 volume ratio. Most preferably, the mixture of methanol and water at 2:1 volume ratio is used. Increased amount of water in the solution contributes to obtaining the expected (S) enantiomer in high selectivity and yield.

Temperature proved to be the critical parameter of the crystallization process. High crystallization selectivity is achieved due to maintaining constant temperature of the crystallization mixture, within the range 20-25° C. When the solution was left at 5° C. for 4-5 h, obtained free base was contaminated with 8.70% (according to HPLC analysis) of (R) enantiomer.

Crystalline solid of diastereoisomeric salt obtained is isolated from the reaction mixture according to standard procedures, for example by filtration or decantation.

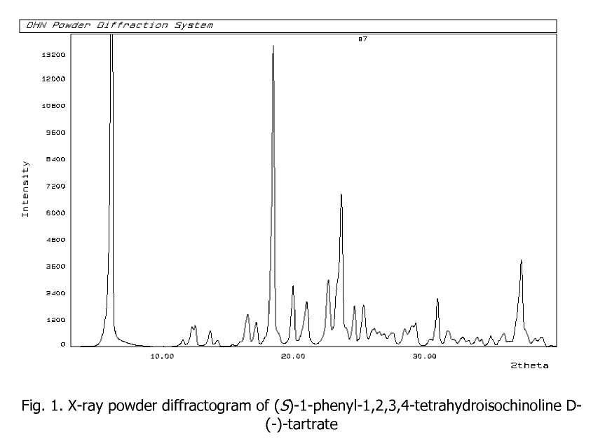

Crystalline salt of (S)-1-phenyl-1,2,3,4-tetrahydroisoquinoline and D-(−)-tartaric acid is characterized by an X-ray powder diffraction pattern (XRPD) substantially as presented in FIG. 1.

At the X-ray diffraction pattern the characteristic peaks are observed presented as the relation of interplanar distances d (\acute{Å}), diffraction angles 2θ (°), and relative intensities, in attitude to the most intensive diffraction peak, I/Iₒ (%), as depicted in Table 1:

TABLE 1
X-ray powder diffraction of (S)-1-phenyl-1,2,3,4-
tetrahydroisoquinoline D-(−)-tartrate

| d, [Å] | 2θ, [°] | I/I₀, [%] |
|---|---|---|
| 14.403 | 6.13 | 100 |
| 7.658 | 11.55 | 1 |
| 7.235 | 12.22 | 3 |
| 7.083 | 12.49 | 3 |
| 6.487 | 13.64 | 3 |
| 6.237 | 14.19 | 1 |
| 5.368 | 16.50 | 5 |
| 5.167 | 17.15 | 4 |
| 4.813 | 18.42 | 49 |
| 4.448 | 19.95 | 10 |
| 4.231 | 20.98 | 7 |
| 3.924 | 22.64 | 11 |
| 3.763 | 23.62 | 25 |
| 3.613 | 24.62 | 7 |
| 3.517 | 25.30 | 7 |
| 2.890 | 30.92 | 8 |
| 2.437 | 36.85 | 4 |

(S)-1-Phenyl-1,2,3,4-tetrahydroisoquinoline is released from diastereoisomeric salt according to standard procedure, e.g. upon treatment with aqueous sodium hydroxide solution in a mixture with organic solvent, for example ethyl acetate. When the phases are separated, aqueous layer is extracted with the same organic solvent, combined organic extracts are washed with water, dried and concentrated under vacuum to dryness.

Use of suitable solvent system, methanol/water, in 1-phenyl-1,2,3,4-tetrahydroisoquinoline enantiomeric resolution, enables isolation of expected (S) enantiomer, characterized by high enantiomeric purity more than 99.6%, preferably 99.8% to 100% in one crystallization step without any need of additional enantiomerical enrichment.

The procedure according to the present invention provides the process for preparation of (S)-1-phenyl-1,2,3,4-tetrahydroisoquinoline, characterized by high enantiomeric purity (determined by HPLC analysis) and high total chemical yield, ranging from 30 to 37%, calculated for the racemic substrate.

The following non-limiting examples are merely illustrative of the preferred embodiment of the present invention and are not to be construed as limiting the invention, the scope of which is defined by the appended claims

### EXAMPLES

### Analytical methods

Enantiomeric purity was determined by HPLC technique, the HPLC device was equipped with chiral column Daicel Chemical Industries LTD, type Chiralcel OD (250×50)×4,6 mm; 10 μm, mobile phase: hexane+propan-2-ol (90+10 v/v, flow 1 mL/min, UV detector, wave length 220 nm) and it was given as enantiomeric excess, calculated according to the equation:

$$ee = \frac{/ S / - / R /}{/ S / + / R /} \times 100 \%$$

where /S/ and /R/ represent peak area of the corresponding isomers, (S) of retention time ca. 11 min and (R) of retention time ca. 19 min.

Melting point was measured by differential scanning calorimetry with Mettler Toledo DSC 822 apparatus, using aluminum melting-pot, with heating speed 10° C./min Melting point value is determined as ‘onset’, which is determined as the cross point of basic line and curve tangents.

X-Ray powder diffraction data were obtained using Rigaku X-ray powder diffractometer type MiniFlex equipped with CuKα detector, λ=1,54056\acute{Å}, using the following measurement parameter:
scanning range 2θfrom 3° to 40°
scanning rate Δω0.5°/mon.
scanning step 0.03°
detector—scintillating counter

Data obtained were worked up and analyzed using DHn-PDS program.

### Example 1

Racemic mixture of 1-phenyl-1,2,3,4-tetrahydroisoquinoline (40 g, 191 mmol) and D-(−)-tartaric acid (28.61 g, 191 mmol, ee 99%) are suspended in methanol (240 mL). The solution is heated to reflux, until the whole amount of solid is completely dissolved. The heating bath is being removed and to the clear solution water (120 mL) is added; the resulting mixture is left at ambient temperature (24° C.) for 24 h. Crystalline solid is collected as residue by filtering the mixture (21.45 g). T₍ₒₙₛₑₜ₎=186.2° C.; [α]²⁵_{D}=−17.02° (c=1%, H₂O). The crystalline solid obtained is suspended in the mixture of 10% NaOH_{aq} (120 mL) and ethyl acetate (50 mL), the solution is stirred at ambient temperature (24° C.) for about 10 min. until the whole amount of solid is dissolved. The reaction mixture is transferred into separatory flask, organic layer is separated and water phase is extracted with ethyl acetate (2×30 mL). Combined organic extracts are washed with water (1×40 mL), dried and condensed under vacuum to dryness. (S)-1-Phenyl-1,2,3,4-tetrahydroisoquinoline is obtained as crystalline solid (12 g, 30%), of enantiomeric excess ee=100%. Chemical purity (HPLC): 99.96%; [α]²⁵_{D}=38.20° (c=1%, CH₂Cl₂).

### Example 2

Following the procedure described in example 1 the enantiomeric resolution of racemate (1 g) with D-(−)-tartaric acid was carried out, employing different mixtures of solvents and crystallization times. The results are collected in Table below.

| | | | Crystal- | | |
|---|---|---|---|---|---|
| | | | lization | Enantiomer | Enantiomer |
| | | Temp. | time | (S) | (R) |
| No. | Solvent | [° C.] | [h] | [HPLC, %] | [HPLC, %] |
| 2 | Methanol | 24 | 2 | 92.3 | 7.50 |
| 3 | Methanol | 24 | 4 | 96.0 | 3.8 |
| 4 | Methanol | 24 | 16 | 67.20 | 32.0 |
| 5 | Methanol-Water | 24 | 3 | 99.66 | 0 |
| | (10:3, v/v) | | | | |
| 6 | Methanol-Water | 5 | 16 | 91.11 | 8.70 |
| | (2:1, v/v) | | | | |
| 7 | Methanol-Water | 24 | 96 | 99.8 | 0 |
| | (2:1, v/v) | | | | |
| 8 | Methanol- | 24 | 1 | 96.98% | 2.90% |
| | i-PrOH(10:3, v/v) | | | | |
| 9 | Methanol- | 24 | 4.5 | 91.48 | 8.3 |
| | i-PrOH (2:1, v/v) | | | | |

### Example 3

Following the procedure described in example 1, the enantiomeric resolution of the racemate (20 g) with D-(−)-tartaric acid in methanol was carried out. After isolation of the I crop of crystals (ee=99.8%), mother liquor was left at 24° C. for 16 h, to yield II crop of crystalline solid (ee=99.25%), after next 16 h at the same temperature III crop (ee=98.4%) was obtained. Crystalline solids collected from the last two crops were combined and recrystallized from methanol—water mixture, resulting crystalline product of enantiomeric excess ee=100% was obtained.

| | | | Crystallization | (S)- | (R)- |
|---|---|---|---|---|---|
| | | Temp. | time | enantiomer | enantiomer |
| No. | Solvent | [° C.] | [h] | [HPLC, %] | [HPLC, %] |
| 10.I | Methanol- | 24 | 24 | 99.80 | 0 |
| | Water | | | | |
| | (2:1, v/v), | | | | |
| | I crop | | | | |
| 10.II | II crop of | 24 | 16 | 97.25 | 2.5 |
| | crystals | | | | |
| 10.III | III crop of | 24 | 16 | 98.4 | 1.4 |
| | crystals | | | | |

1. A process for preparation of (S)-1-phenyl-1,2,3,4-tetrahydroisoquinoline, comprising reacting 1-phenyl-1,2,3,4-tetrahydroisoquinoline with D-(−)-tartaric acid in a solvent system, comprising methanol and water, allowing the resulting crystallization mixture to crystallize, and releasing (S)-1-phenyl-1,2,3,4-tetrahydroisoquinoline from a crystalline diastereoisomeric salt according to standard procedures.

2. The process according to claim 1, wherein the solvent system comprises methanol and water at 3.3:1 to 1:1 volume ratio.

3. The process according to claim 2, wherein the solvent system comprises methanol and water at 2:1 volume ratio.

4. The process according to claims 1, wherein the crystallization mixture temperature is 20-25° C.

5. The process according to claim 1, wherein the (S)-1-phenyl-1,2,3,4-tetrahydroisoquinoline is obtained in enantiomeric purity higher than 99.5%.

6. The process according to claim 1, wherein the (S)-1-phenyl-1,2,3,4-tetrahydroisoquinoline is obtained in chemical purity (analyzed by HPLC) higher than 99.5%,

7. A crystalline (S)-1-phenyl-1,2,3,4-tetrahydroisoquinoline D-(−)-tartrate, characterized by X-ray powder diffraction pattern, which is represented as the relation of interplanar distances d (\acute{Å}), diffraction angles 2θ(°), and relative intensities, in attitude to the most intensive diffraction peak, I/Iₒ (%):

| d, [Å] | 2θ, [°] | I/I₀, [%] |
|---|---|---|
| 14.403 | 6.13 | 100 |
| 7.658 | 11.55 | 1 |
| 7.235 | 12.22 | 3 |
| 7.083 | 12.49 | 3 |
| 6.487 | 13.64 | 3 |
| 6.237 | 14.19 | 1 |
| 5.368 | 16.50 | 5 |
| 5.167 | 17.15 | 4 |
| 4.813 | 18.42 | 49 |
| 4.448 | 19.95 | 10 |
| 4.231 | 20.98 | 7 |
| 3.924 | 22.64 | 11 |
| 3.763 | 23.62 | 25 |
| 3.613 | 24.62 | 7 |
| 3.517 | 25.30 | 7 |
| 2.890 | 30.92 | 8 |
| 2.437 | 36.85 | 4 |

8. The crystalline (S)-1-phenyl-1,2,3,4-tetrahydroisoquinoline D-(−)-tartrate according to claim 7, characterized by X-ray powder diffraction pattern as depicted in FIG. 1.

9. A process for the preparation of solifenacin or salts thereof, comprising reacting of quinuclidinol and carbamoyl derivative of the (S)-1-phenyl-1,2,3,4-tetrahydroisoquinoline, prepared according to the process of claim 1, 2 or 4, with a good leaving group.

10. The process according to claim 9, wherein the good leaving group includes a chloride anion, one or more lower alkoxides, a phenoxide, 1H-imidazol-1-yl, 2,5-dioxopyrrolidin-1-yloxy or a 3-methyl-1H-imidazol-3-ium-1-yl group.

11. A process for the preparation of solifenacin or salts thereof, comprising the condensation of the (S)-1-phenyl-1,2,3,4-tetrahydroisoquinoline, prepared according to the process of claim 1, 2 or 4, with activated quinucidinol derivative.

12. The process according to claim 5, wherein the (S)-1-phenyl-1,2,3,4-tetrahydroisoquinoline is obtained in enantiomeric purity of 99.8% to 100%.

13. The process according to claim 6, wherein the (S)-1-phenyl-1,2,3,4-tetrahydroisoquinoline is obtained in chemical purity (analyzed by HPLC) of equal to or higher than 99.8%.

14. The process according to claim 2, wherein the crystallization mixture temperature is 20-25° C.
